(19) United States
(12) Patent Application Publication
(10) Pub. No.: US 2012/0197054 A1
(43) Pub. Date: Aug. 2, 2012

(54) PROCESS AND APPARATUS FOR DEHYDRATING ALKANES WITH EQUALIZATION OF THE PRODUCT COMPOSITION

(75) Inventors: Helmut Gehrke, Bergkamen (DE); Rolf Schwass, Neubeckum (DE); Max Heinritz-Adrian, Muenster (DE); Oliver Noll, Castrop-Rauxel (DE); Sascha Wenzel, Bochum (DE)

(73) Assignee: THYSSENKRUPP UHDE GMBH, Dortmund (DE)

(21) Appl. No.: 13/386,588

(22) PCT Filed: Jul. 16, 2010
(86) PCT No.: PCT/EP10/04348

§ 371 Date: Mar. 5, 2012

(30) Foreign Application Priority Data
Jul. 22, 2009 (DE) 10 2009 034 464.0

(51) Int. Cl. C07C 5/48

(52) U.S. Cl. 585/501

(57) ABSTRACT

A process for the dehydrogenation of alkanes. In several reactors of the adiabatic, allothermal or isothermal type or combinations thereof a gaseous alkane-containing material stream is passed through a catalyst bed in continuous operating mode. The gas stream produced contains an alkene, hydrogen and a non-converted alkane. In order to achieve a constant product composition, at least one of the process parameters of temperature, pressure or steam/hydrocarbon ratio is recorded in the form of measured values at one or several points of at least one of the reactors, where at least one of the process parameters is selectively controlled and influenced such that the composition of the product gas at the outlet of one reactor remains constant throughout the operating period.

The invention relates to a process for the dehydrogenation of alkanes with constant product composition by passing an alkane over a suitable catalyst, a gas stream being formed which contains an alkene, hydrogen and a non-converted alkane. As the dehydrogenation of alkanes belongs to the group of reversible equilibrium reactions, the chemical equilibrium is reached during the reaction after a specific residence time under ideal catalyst conditions. Consistency in the product composition, i.e. a constant content of alkene, alkane and hydrogen in the product gas is achieved by shifting the chemical equilibrium to the desired direction by modifying the process parameters.

The dehydrogenation of alkanes takes place on a suitable catalyst. The activity of the catalyst gradually decreases while the reaction conditions remain the same, thus causing permanent changes of the product composition at the reactor outlet during a production cycle provided the process parameters remain unchanged. Failures in the downstream plant sections may occur on account of the permanently changing product composition. The rectification columns, for example, are susceptible to variations in the concentration of the feedstock stream.

U.S. Pat. No. 5,243,122 A describes a process for the dehydrogenation of light alkanes in an allothermal reformer, the temperature of the catalyst bed being controlled and slightly increased during the reaction such that the composition of the reactor effluent remains constant during the reaction. This measure delays the decrease in the catalyst activity such that the composition of the product stream and particularly the alkene/alkane ratio contained therein remain constant during operation. Thermal reaction control is provided by a special valve control system for fuel gas supply. However, the reformers are arranged in parallel, other influencing factors except for the temperature have not been considered.

Normally, after some time during the reaction, carbon-bearing deposits occur on the catalyst, drastically reducing the alkane conversion rate. For this reason the reaction is carried out in cycles. After a specific reaction time the reaction is stopped and an oxygen-containing gas which may also contain water vapour is passed over the catalyst. The carbon-bearing deposits are oxidised by this gas such that the catalyst is no longer covered and the reaction can start again.

The objective of the invention is therefore to develop an alkane dehydrogenation process which ensures constant product composition at the reactor outlet throughout the entire operating period.

The objective is achieved by passing a gaseous alkane-containing material stream in continuous operating mode through a catalyst bed in several reactors of adiabatic, allothermal or isothermal type or combinations thereof, a gas stream being produced which contains an alkene, hydrogen and a non-converted alkane, and by
recording at least one of the process parameters of temperature, pressure or steam/hydrocarbon ratio in the form of measured values at one or several points of at least one of the reactors,
selectively influencing at least one of the process parameters is such that the composition of the product gas at the outlet of at least one reactor remains constant throughout the operating period.

At one or several points of a reactor measured values of temperature, pressure or steam/hydrocarbon ratio can be taken, then the process parameters can be controlled and influenced selectively by means of controllers such that the composition of the product gas at the end of the reactor system remains constant throughout the operating period.

In embodiments of the invention it is envisaged that two to ten identical or different reactor types are operated as an interlinked system. However, two to four reactors are preferred for economical reasons. The reactors may be of the allothermal, adiabatic or isothermal types. Of course, the reactors may also be combined with different types to achieve a corresponding effectiveness and economic efficiency. To achieve a constant product composition, the process parameters of temperature, pressure and steam/hydrocarbon ratio may be influenced selectively. The temperature can be controlled in at least one of the reactors by the fuel gas/oxygen supply and a suitable temperature sensor. In the same way the pressure in the reactor can be controlled by means of a control valve in the product gas discharge. The steam/hydrocarbon ratio in the reactor is determined by the supplied amounts of steam and gaseous hydrocarbon, this action being preferred to take place in the first of the reactors.

In further embodiments of the invention an analyser for measuring the composition of the product gas is deployed. The analyser may be, for example, a gas chromatograph. With the specified target value of temperature, pressure or steam/hydrocarbon ratio the composition of the product gas is determined with the aid of the analyser. As a result, both individual and combined process parameters can be influenced such that the desired constant product composition can be achieved. The same can also be achieved by specifying a time-variable function, as for example, a ramp function, by means of a process control system.

In further embodiments of the invention the use of the inventive process for the production of alkenes from alkanes is also claimed, particularly the use of the process for the dehydrogenation of propane to propene, of n-butane to n-butenes and butadiene, of isobutane to isobutene, or mixtures thereof and for the dehydrocyclisation of alkanes to aromatic hydrocarbons. However, any alkane or any hydrocarbon can be dehydrogenated that is dehydrogenable by a state-of-the-art dehydrogenation process.

The invention is illustrated by some examples, an allothermal reactor being considered as embodiment for the dehydrogenation of propane to propene in order to present the inventive process. The reactor is operated with the following process parameters: inlet temperature: 510° C., temperature difference ΔT between inlet and outlet: 75K, outlet pressure p: 6.0 bar, molar steam/hydrocarbon ratio STHC: 3.5.

### EXAMPLE 1

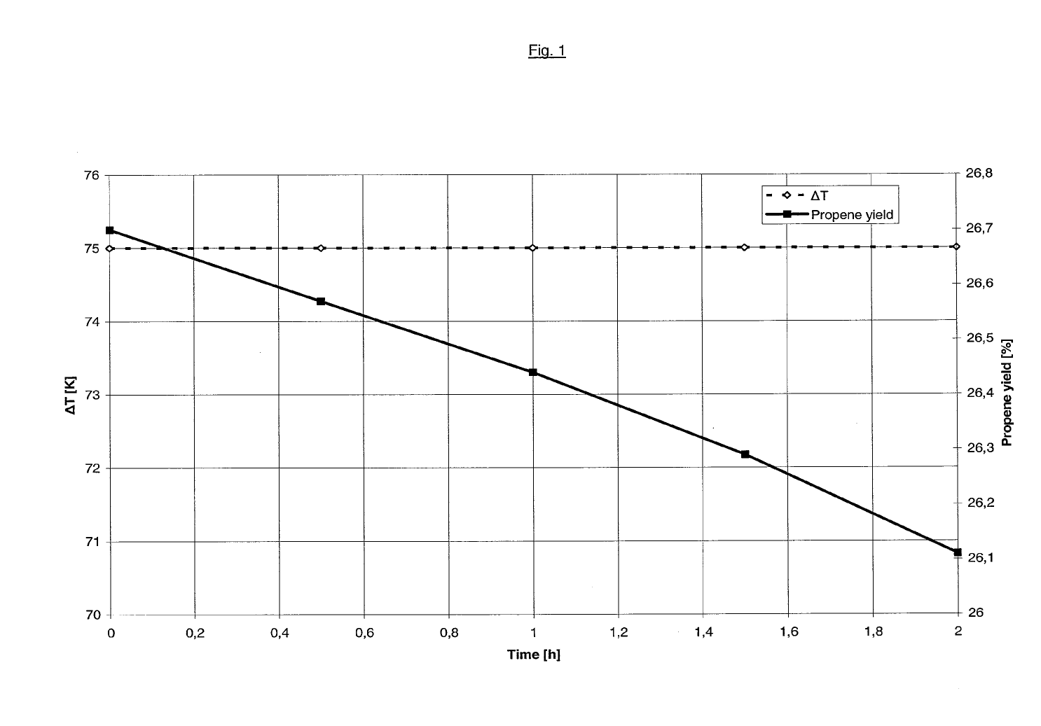

As shown in FIG. 1, the propene yield decreases from initially 26.7% to 26.1% if the process parameters are not changed.

### EXAMPLE 2

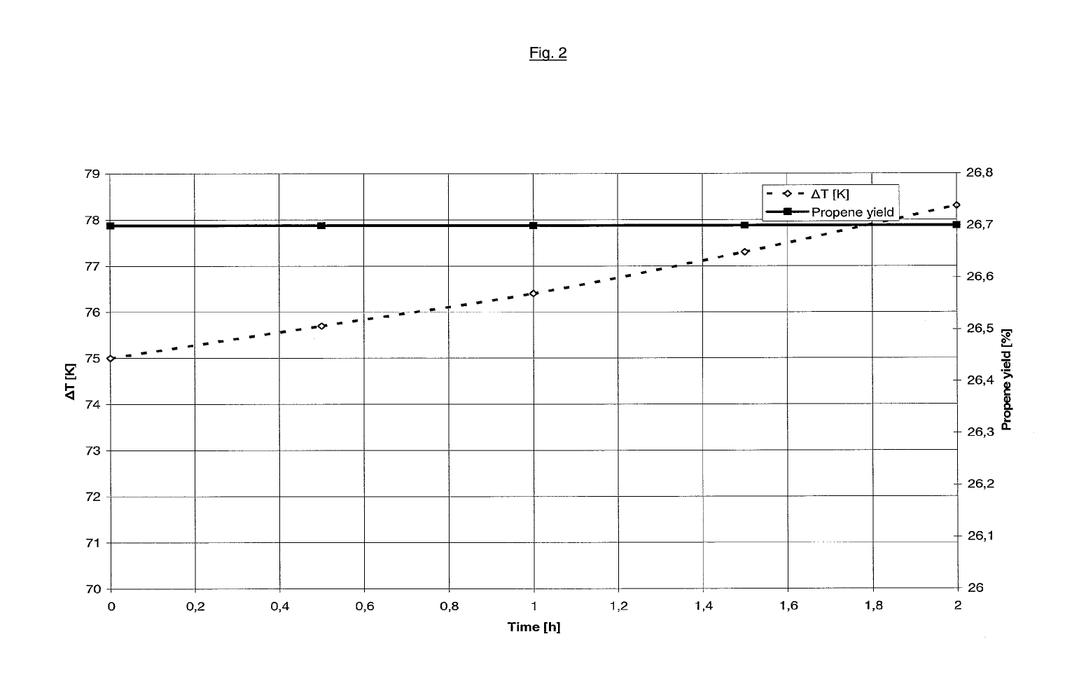

As shown in FIG. 2, the propene yield is kept constant at 26.7% if the temperature difference ΔT is increased over the entire cycle. All other parameters remain unchanged as in example 1.

### EXAMPLE 3

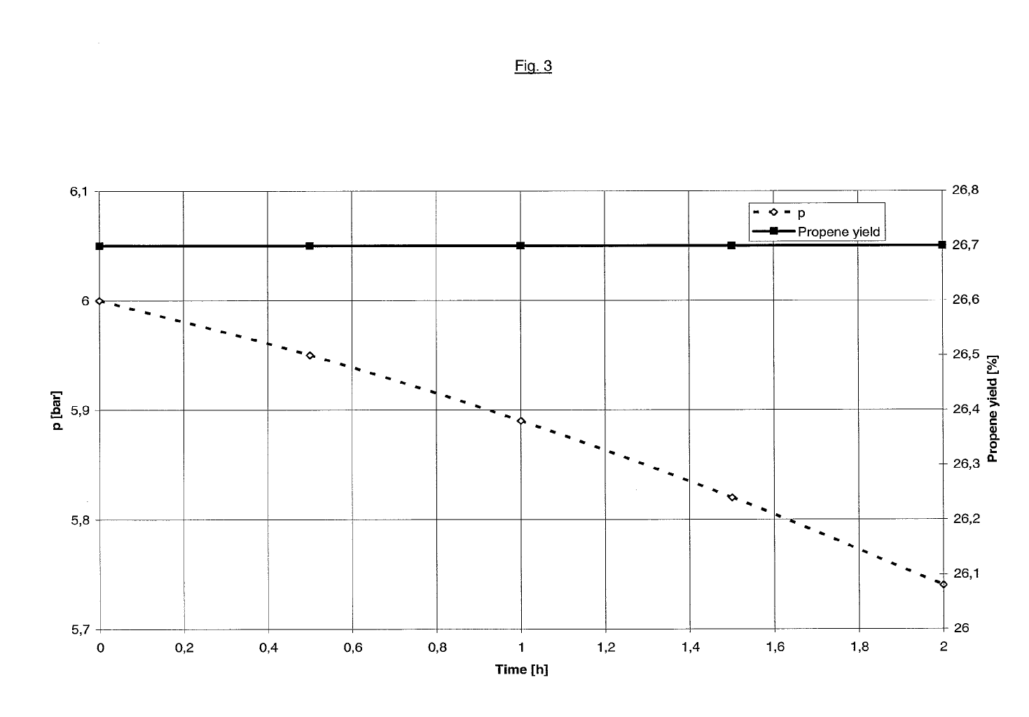

As shown in FIG. 3, the propene yield is kept constant at 26.7% if the outlet pressure p is reduced over the entire cycle. All other parameters remain unchanged as example 1.

### EXAMPLE 4

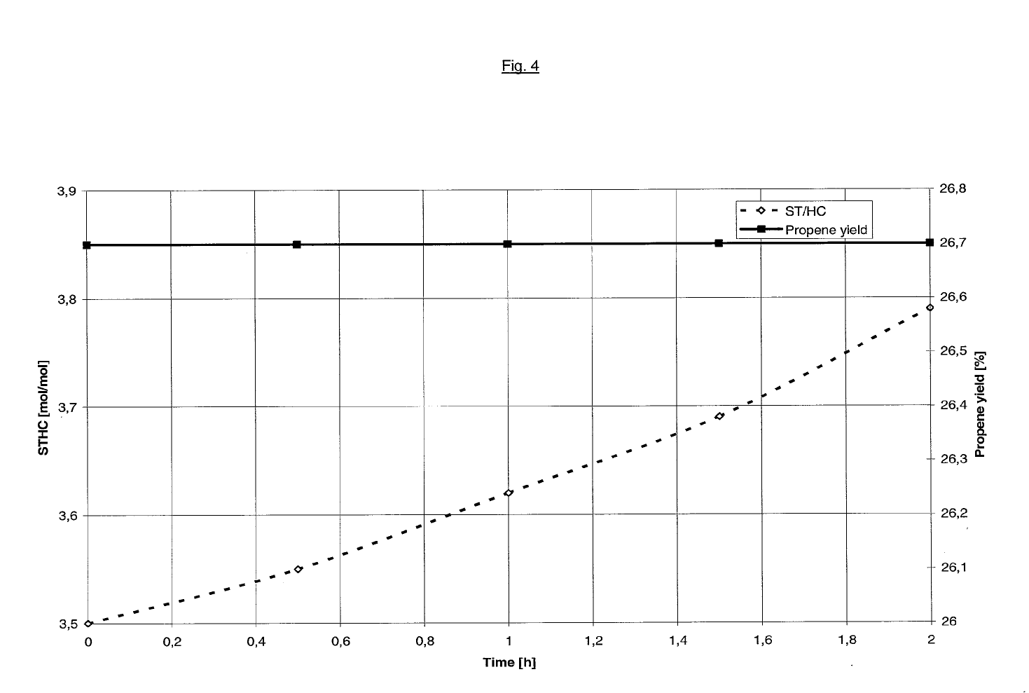

As shown in FIG. 4, the propene yield is kept constant at 26.7% if the steam/hydrocarbon ratio (STHC) is increased over the entire cycle. All other parameters remain unchanged as example 1.

### EXAMPLE 5

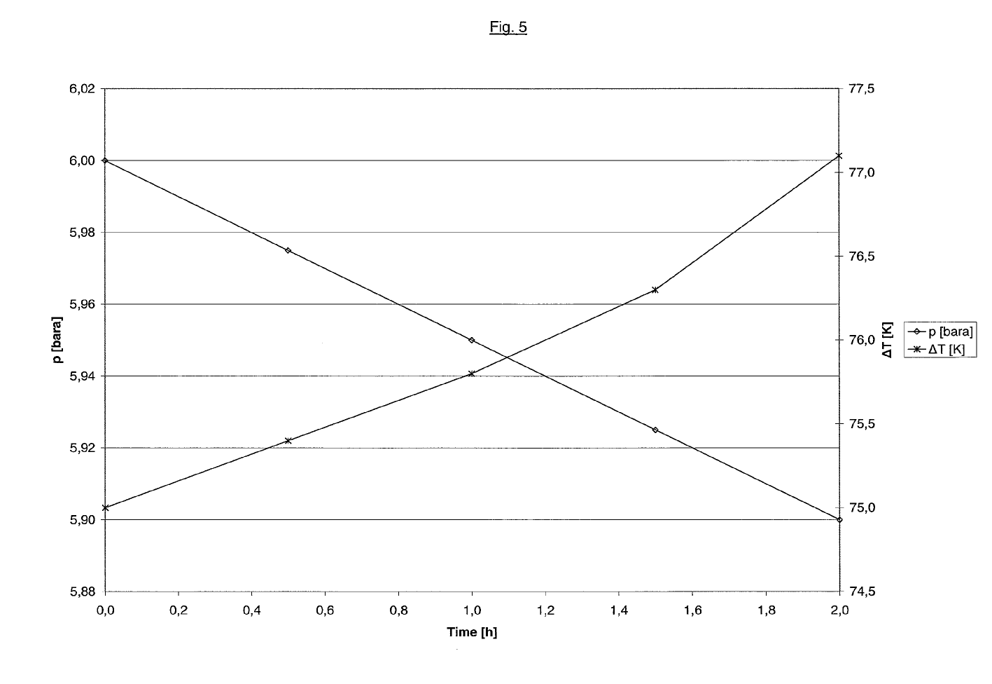

As shown in FIG. 5, the pressure in this example is constantly reduced by 0.05 bar/h over the entire cycle and the temperature difference ΔT at the same time slightly increased to achieve a uniform propene yield. Frequently, in practice, an isolated reduction of the outlet pressure p over the time is not arbitrarily feasible (as in example 3) because the subsequent process step, e.g. raw gas compression, requests a specific inlet pressure. It is therefore advisable to influence several process parameters at the same time to achieve the desired constant product gas composition.

Table 1 summarises the examples which show the obvious effects of the influence of the process parameters on the product gas composition.

TABLE 1
Overview of parameters set

| Example | t [h] | | 0.0 | 0.5 | 1.0 | 1.5 | 2.0 |
|---|---|---|---|---|---|---|---|
| Example 1 | unchanged | propene | 26.70 | 26.57 | 26.44 | 26.29 | 26.11 |
| | parameters | yield | | | | | |
| | | (mol. %) | | | | | |
| Example 2 | parameter | ΔT (K) | 75.0 | 75.7 | 76.4 | 77.3 | 78.3 |
| | change | | | | | | |
| Example 3 | parameter | p (bar) | 6.00 | 5.95 | 5.89 | 5.82 | 5.74 |
| | change | | | | | | |
| Example 4 | parameter | STHC¹⁾ | 3.50 | 3.55 | 3.62 | 3.69 | 3.79 |
| | change | | | | | | |
| Example 5 | parameter | p (bar) | 6.00 | 5.98 | 5.96 | 5.93 | 5.90 |
| | change | ΔT (K) | 75 | 75.4 | 75.8 | 76.3 | 77.1 |

¹⁾STHC: molar steam/hydrocarbon ratio

The invention is explained in the following on the basis of the drawings.

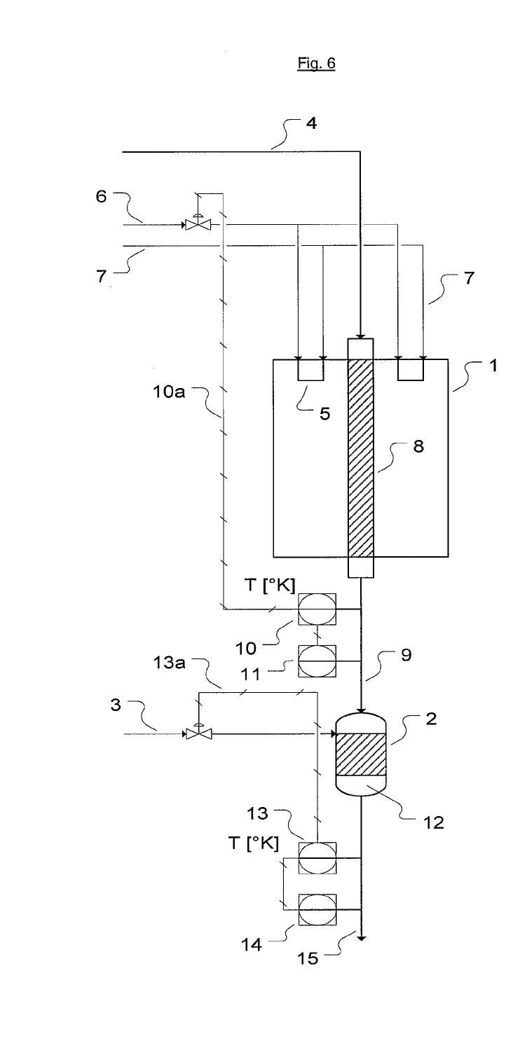

FIG. 6: An apparatus consisting of an allothermal and an adiabatic reactor connected in series with a temperature control system.

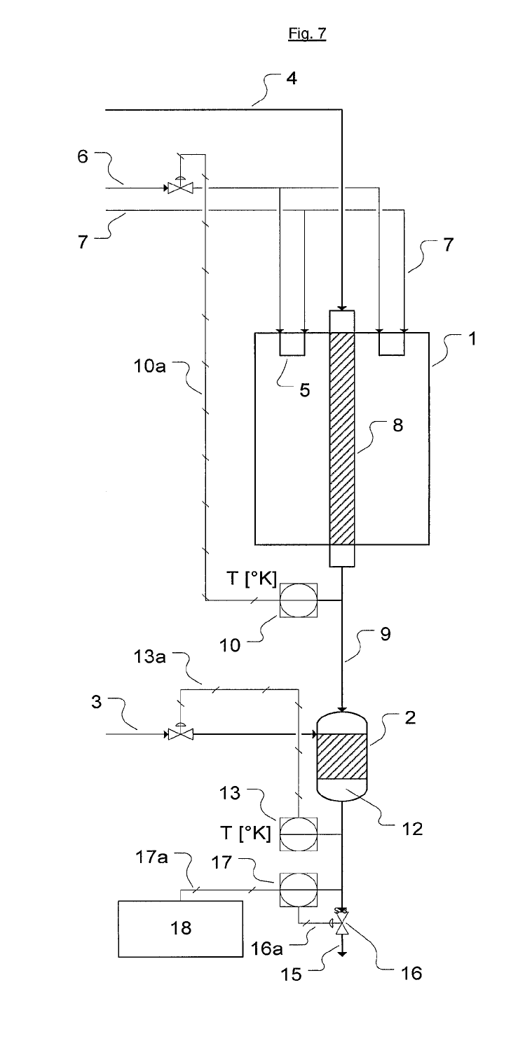

FIG. 7: An apparatus consisting of an allothermal and an adiabatic reactor connected in series including a temperature control system and a pressure control system.

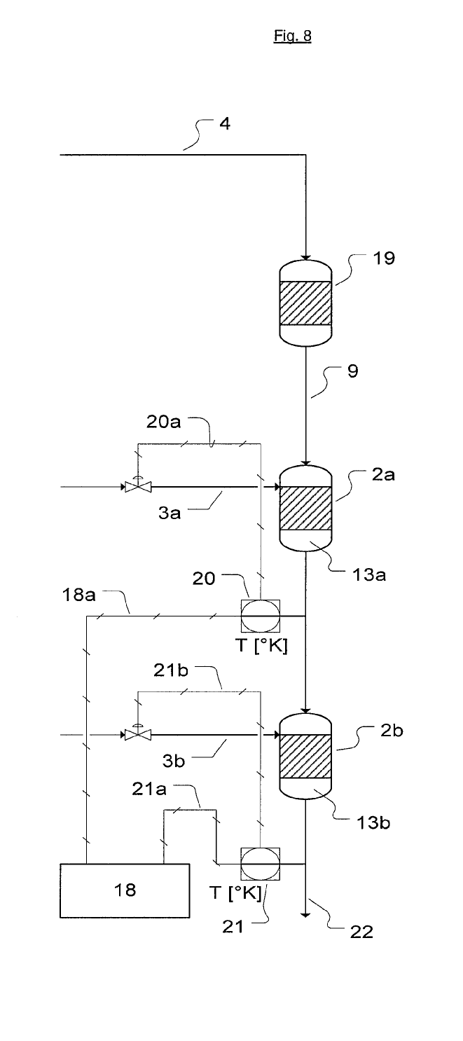

FIG. 8: An apparatus consisting of adiabatic reactors connected in series including a temperature and a pressure control system by means of a process control system.

FIG. 6 shows an apparatus consisting of two series-connected reactors of allothermal (1) and adiabatic (2) type with oxygen supply (3). The reaction gas (4) is fed to the allothermal reactor (1). The heating is carried out by means of the burners (5) which are operated with a fuel gas (6) and an oxygen-containing gas (7). In the reactor (1) a closed piping system (8) is provided in which there is a catalyst and the reaction takes place. At the outlet of the first reaction system (1) a temperature measuring instrument (10) and an analyser (11) are connected. The fuel gas supply is controlled by means of the temperature measuring instrument (10) and the electrical control lines (10a) such that the measured values of the analyser (11) always show the desired same content of alkene in the product gas (9). The product gas (9) from the reactor system (1) is then mixed with an oxygen-containing gas (3) and fed to the adiabatic reactor (2). In this reactor there is also a closed piping system for dehydrogenation and hydrogen oxidation (12) which contains a catalyst and where hydrogen oxidation and further dehydrogenation take place. At the outlet of the second reactor there is also a temperature measuring instrument (13) and an analyser (14). The oxygen supply is controlled by means of the temperature measuring instrument (13) and the electrical control lines (13a) such that the measured values of the analyser (14) always show the desired same content of alkene in the product gas (15).

FIG. 7 shows an apparatus which also consists of a first allothermically operated reactor (1) and a second adiabatically operated reactor (2) with oxygen supply (3). The temperature is measured at the outlet of the first reaction system (9) by means of a temperature measuring instrument (10) and controlled in dependency of the fuel gas supply and the oxygen supply (6,7) by means of electric measuring signals (10a). In this way, a constant temperature can be adjusted in the first reaction system. In this device the product composition is only controlled at the outlet of the second reaction system (15). This is done by means of an analyser (17) at the outlet of the second reaction system, the said analyser measuring the pressure by means of a pressure control valve (16) on the reactor of the second reaction system (2) and forwarding them by means of electrical control lines (16a, 17a) to a process control system (18). The temperature of the reactor (2) is controlled via the electrical control line (13a) and the oxygen supply (3). The process control system (18) calculates the required pressure settings and performs its control task by means of the electric measuring signals (17a) and the pressure control valve (16) at the outlet of the reactor system such that the composition of the product gas (15) obtained at the outlet of the second reactor (2) is always the same.

FIG. 8 shows an apparatus consisting of three series-connected adiabatic reactors (19, 2a, 2b) with oxygen supply (3a, 3b). The reaction in the first reactor (19) runs adiabatically such that a steadily changing product composition is achieved at the outlet of the reaction system (9). In the reactors (2a, 2b) a selective hydrogen oxidation is carried out. At the outlet of the second reactor (2a) a temperature measuring instrument (20) is provided which controls the reactor (2a) via the electrical control lines (20a) and the oxygen supply (3a). The measured values of the temperature measuring instrument (20) are forwarded to a process control system (18) via the electrical control lines (18a). This gives a product gas composition at the outlet of the reactor (2a). At the outlet of the third reactor (2b) another temperature measuring instrument (21) is positioned which controls the connected reactor via the electrical control lines (21b) and the oxygen supply (3a). The temperature measuring instrument (21) forwards the measured values to the process control system (18) via the electrical control line (21a). This gives a desired constant product gas composition at the outlet of the third reaction system (22).

### LIST OF REFERENCES USED

1 Allothermally heated reactor
2 Adiabatically operated reactor
3 Oxygen supply
3a Oxygen supply
3b Oxygen supply
4 Reaction gas
5 Burner
6 Fuel gas
7 Oxygen-containing gas
8 Closed piping system for dehydrogenation reaction
9 Product gas from first reaction system
10 Temperature measuring instrument
10a Electrical control line
11 Analyser for determining the product gas composition
12 Closed piping system for dehydrogenation and hydrogen oxidation
13 Temperature measuring instrument
13a Electrical control line
14 Analyser for determining the content of alkene in the product gas
15 Product gas
16 Pressure control valve
16a Electrical control line
17 Analyser
17a Electrical control line
18 Process control system
18a Electrical control line
19 Adiabatically operated reactor
20 Temperature measuring instrument
20a Electrical control line
21 Temperature measuring instrument
21a Electrical control line
21b Electrical control line
22 Product gas

1. Process for the dehydrogenation of alkanes with constant product composition, in which
a gaseous alkane-containing material stream is passed in continuous operating mode through a catalyst bed in several reactors of the adiabatic, allothermal or isothermal type or combinations thereof, a gas stream being produced which contains an alkene, hydrogen and a non-converted alkane,
characterised in that
at least one of the process parameters of temperature, pressure or steam/hydrocarbon ratio is recorded in the form of measured values at one or several points of at least one of the reactors,
at least one of the process parameters is selectively controlled and influenced such that the composition of the product gas at the outlet of at least one of the reactors remains constant throughout the operating period.

2. Process for the dehydrogenation of alkanes with constant product composition according to claim 1, characterised in that two to ten, preferably two to four reactors of different types are operated as an interlinked system.

3. Process for the dehydrogenation of alkanes with constant product composition according to claim 1, characterised in that two to ten, preferably two to four reactors of the same type are operated as an interlinked system.

4. Process for the dehydrogenation of alkanes with constant product composition according to one of preceding claims 1 to 3, characterised in that the temperature in one of the reactors is controlled by the fuel gas supply and a temperature sensor.

5. Process for the dehydrogenation of alkanes with constant product composition according to one of preceding claims 1 to 3, characterised in that the temperature in one of the reactors is controlled via the oxygen supply by means of a temperature sensor.

6. Process for the dehydrogenation of alkanes with constant product composition according to one of preceding claims 1 to 5, characterised in that the pressure in at least one of the reactors is controlled via the product gas discharge by means of a control valve.

7. Process for the dehydrogenation of alkanes with constant product composition according to one of preceding claims 1 to 6, characterised in that the steam/hydrocarbon ratio in at least one of the reactors is controlled by the supplied amounts of steam and gaseous hydrocarbon.

8. Process for the dehydrogenation of alkanes with constant product composition according to claim 7, characterised in that the steam/hydrocarbon ratio in the first of the reactors is controlled by the supplied amounts of steam and gaseous hydrocarbon.

9. Process for the dehydrogenation of alkanes with constant product composition according to one of preceding claims 1 to 8, characterised in that the process parameters in at least one reactor are influenced in dependency of the values measured for the product gas composition by an analyser.

10. Process for the dehydrogenation of alkanes with constant product composition according to one of preceding claims 1 to 8, characterised in that the process parameters in at least one reactor are influenced by specifying a time-variable function by means of a process control system.

11. Process for the dehydrogenation of alkanes with constant product composition according to one of preceding claims 1 to 10, characterised in that several process parameters are influenced simultaneously.

12. Use of the process according to one of preceding claims 1 to 11 for the dehydrogenation of propane to propene.

13. Use of the process according to one of preceding claims 1 to 11 for the dehydrogenation of n-butane to n-butenes and butadiene.

14. Use of the process according to one of preceding claims 1 to 11 for the dehydrogenation of iso-butane to iso-butene.

15. Use of the process according to one of preceding claims 1 to 11 for the dehydrocyclisation of alkanes to aromatic hydrocarbons.
